(19) United States
(12) Patent Application Publication
(10) Pub. No.: US 2004/0006362 A1
(43) Pub. Date: Jan. 8, 2004

(54) Uniaxial multifilar vaso-occlusive device with high stretch resistance and low buckling strength

(76) Inventors: Dean Schaefer, Laguna Hills, CA (US); Horacio Almazan, Rancho Santa Margarita, CA (US)

Correspondence Address: Howard J. Klein, KLEIN, O'NEILL & SINGH, 2 Park Plaza, Suite 510, Irvine, CA, 92614, US

(21) Appl. No.: 10/188,492

(22) Filed: Jul. 2, 2002

(51) Int. Cl. A61M 29/00

(52) U.S. Cl. 606/200

(57) ABSTRACT

A multifilar vaso-occlusive implant device includes a plurality of elongate filars conjointly wound into helical coils having respective windings arranged in an alternating, uniaxial adjacency with each other. The respective windings of the coils may have the same or different mean diameters and pitches, and the materials, cross-sectional shapes and areas of the respective filars of the coils may be the same or variegated to achieve a desirable increase in the axial stretch resistance, and reductions in the respective axial bending and buckling resistances of the device. A rounded obturator tip may advantageously be attached at the distal end of the device, and a coupling element for releasably coupling the device to a delivery mechanism may advantageously be attached to the proximal end of the device. A flexible tube or porous sponge may be disposed in the axial lumen of the device and loaded with a bio-active agent for delivery to a patient via implantation of the device in the patient.

### CROSS-REFERENCE TO RELATED APPLICATIONS

This application is related to U.S. patent application Ser. No. ______, filed ______.

### FEDERALLY SPONSORED RESEARCH OR DEVELOPMENT

Not Applicable

### BACKGROUND OF THE INVENTION

This invention is related to the field of vascular occlusion in general, and in particular, to elongated, recurvate, helically wound, multifilar vaso-occlusive devices that have a high resistance to axial stretching and kinking, yet a low resistance to axial bending and buckling.

Vaso-occlusive devices are typically used within the vasculature of the human body to block the flow of blood through a vessel by forming an embolus therein. Vaso-occlusive devices are also used to form an embolus within an aneurysm stemming from the vessel. Vaso-occlusive devices can be formed of one or more elements, generally delivered into the vasculature via a catheter or similar mechanism.

The embolization of blood vessels is desired in a number of clinical situations. For example, vascular embolization has been used to control vascular bleeding, to occlude the blood supply to tumors, and to occlude vascular aneurysms, particularly intracranial aneurysms. In recent years, vascular embolization for the treatment of aneurysms has received much attention. Several different treatment modalities have been employed in the prior art.

One approach that has shown promise is the use of small, thrombogenic helical coils, or “microcoils.” These microcoils may be made of a biocompatible metal alloy (typically platinum and tungsten) or a suitable polymer. If made of metal, the microcoil may be provided with Dacron fibers to increase thrombogenicity. The microcoil is deployed through a microcatheter to the vascular site. Examples of such microcoils are disclosed in the following U.S. Pat. Nos. 4,994,069—Ritchart et al.; 5,133,731—Butler et al.; 5,226,911—Chee et al.; 5,312,415—Palermo; 5,382,259—Phelps et al.; 5,382,260—Dormandy, Jr. et al.; 5,476,472—Dormandy, Jr. et al.; 5,578,074—Mirigian; 5,582,619—Ken; 5,624,461—Mariant; 5,645,558—Horton; 5,658,308—Snyder; and 5,718,711—Berenstein et al.

A specific type of microcoil that has achieved a measure of success is the Guglielmi Detachable Coil (“GDC”), described in U.S. Pat. No. 5,122,136—Guglielmi et al. The GDC employs a single, platinum-wire helical coil fixed to a stainless steel delivery wire by a solder connection. During its emplacement in an aneurysm, the microcoil loses its elongated linear shape and recurves back upon itself This recurvation of the microcoil occurs either because of a recurvate, “secondary memory” formed in the microcoil during its manufacture, or as a result of axial buckling and bending of the microcoil when its distal end contacts the wall of the aneurysm, or both.

Several microcoils of different diameters and lengths can be packed into an aneurysm until it is substantially filled with a porous embolus of recurved microcoils. After each microcoil is placed inside the aneurysm, an electrical current is applied to the delivery wire, which electrolytically disintegrates the solder junction, thereby detaching the microcoil from the delivery wire. The application of the current also creates a positive electrical charge on the microcoil, which attracts negatively-charged blood cells, platelets, and fibrinogen, thereby increasing the thrombogenicity of the microcoil. The microcoils thus create and hold a thrombus within the aneurysm, thereby inhibiting its displacement and fragmentation.

One of the advantages of the GDC and other microcoil techniques is the ability to with-draw and relocate a microcoil if it migrates from its desired location. However, while the microcoil-type vaso-occlusive devices of the prior art can be withdrawn and relocated, they are prone to an inelastic axial elongation (i.e., stretching) and kinking during deployment, especially if a partial retrieval is needed to reposition the device. Such deformation of the vaso-occlusive device can result in the need for the complete retrieval of the damaged device, and the insertion of a new one.

Inelastic axial compression is typically not a problem in vaso-occlusive microcoils, since the devices are typically fabricated and inserted in a close-wound condition, i.e., with the adjacent coils of the device substantially in abutment with one another. Rather, a problem that can be encountered with prior art vaso-occlusive microcoils relates to their resistance to axial buckling. Thus, during emplacement of the device, its distal end or tip will eventually encounter the wall of the aneurysm or other cavity in which the device is being emplaced, and this is typically so even with devices having a built-in recurvate memory. It is therefore very desirable that the device will immediately buckle axially when this contact occurs, such that the distal tip of the device is deflected parallel to or away from the wall of the cavity, rather than penetrating into or through the wall, which can occur if the device has too much resistance to axial buckling and is inserted with sufficient force.

To address the above inelastic stretching problem, various efforts have been made to increase the resistance to axial stretching of a vaso-occlusive device. These typically involve incorporating one or more axial strands through the center of the device that essentially tie the two opposite ends of the device together, and thereby prevent them from being pulled apart axially during retrograde axial movement of the device. Examples of these efforts can be found in U.S. Pat. Nos. 6,159,165—Ferrera et al.; 6,013,084—Ken et al.; 5,853,418—Ken et al.; 5,582,619—Ken. While each of these addresses the device stretching problem to some extent, they do not address the resistance to tip deflection or axial buckling problem described above, and in most instances, can exacerbate it, because the inelastic strands or members incorporated into the core of the device invariably increase its bending resistance to some extent.

There has thus been a long-felt, but as yet unsatisfied need for a microcoil type of vaso-occlusive device that has a substantially increased resistance to axial stretching and kinking, yet a substantially reduced resistance to axial bending and buckling, for the effective occlusive treatment of aneurysms and other body cavities.

### SUMMARY OF THE INVENTION

In accordance with the present invention, a vaso-occlusive microcoil implant device is provided that has a substantially increased resistance to axial stretching and kinking, yet substantially the same or a reduced resistance to axial bending, as well as a reduced resistance to axial buckling, during both deployment and repositioning within the vasculature.

The novel vaso-occlusive device comprises a multiplicity of filars conjointly wound into elongate helical coils having their respective windings arranged in alternating, uniaxial adjacency. In one exemplary embodiment, each coil comprises an elongate, resilient filar having a uniform cross-sectional shape, area and material, and these may be varied from filar to filar, together with the number of filars and the diameter and pitch of their respective windings, to achieve desirable mechanical properties of the device, including an increased resistance to axial stretching, and a reduction in the resistance to axial bending and buckling of the device.

In another exemplary embodiment, the filar of at least one of the coils comprises a metal, e.g., platinum or an alloy thereof In another possible embodiment, the filar of at least one spring may comprise a polymer.

Advantageously, a rounded ball tip is attached at a distal end of the device to serve as an atraumatic obturator, and a coupler may be provided at a proximal end of the device for releasably coupling the device to a delivery mechanism, such as a delivery wire.

In yet another advantageous embodiment, a flexible tube or a porous sponge may be disposed within the axial lumen of the device, and bio-active agents, such as medications in the form of a powder or a liquid, can be loaded into the tube or sponge for delivery to the patient via the device.

In one possible embodiment of a method for making the device, the plurality of coils are conjointly wound around a support mandrel, and in another possible method, conjointly wound without a support mandrel using a deflection winder.

The above and other features and advantages of the present invention will be more readily apparent from the detailed description of the embodiments set forth below, especially when taken in conjunction with the figures of the accompanying drawings.

### BRIEF DESCRIPTION OF THE DRAWINGS

FIG. 1 is a partial cross-sectional elevation view of a conventional microcoil-type vaso-occlusive implant device in accordance with the prior art;

FIG. 2 is an enlarged cross-sectional axial view of the conventional device of FIG. 1, as revealed by the section taken therein along the lines 2-2;

FIG. 3 is an enlarged cross-sectional elevation view of the conventional device of FIG. 1 showing the relationship of the respective windings thereof, as revealed by the section taken in FIG. 2 along the lines 3-3;

FIG. 4 is a partial cross-sectional elevation view of one exemplary preferred embodiment of a microcoil-type vaso-occlusive device in accordance with the present invention;

FIG. 5 is an enlarged cross-sectional axial view of the novel device of FIG. 4, as revealed by the section taken therein along the lines 5-5;

FIG. 6 is an enlarged cross-sectional elevation view of the novel device of FIG. 4 showing the relationship of the windings thereof, as revealed by the section taken in FIG. 5 along the lines 6-6;

FIG. 7A is a schematic cross-sectional elevation view of a single helical coil having its windings in uniaxial adjacency in accordance with microcoil-type vaso-occlusive devices of the prior art, and being loaded in axial tension;

FIG. 7B is a schematic cross-sectional elevation view of a helical coil similar to that shown in FIG. 7A, except having its windings spaced at twice the pitch of the former, and being loaded in axial tension;

FIG. 7C is a schematic cross-sectional elevation view of a plurality of the double-pitched helical coils shown in FIG. 7B being loaded in parallel, axial tension;

FIG. 7D is a schematic cross-sectional elevation view of the plurality of the double-pitched helical coils shown in FIG. 7C combined such that their respective windings are in alternating, uniaxial adjacency and being loaded in parallel, axial tension;

FIG. 8A is a schematic cross-sectional elevation view of a single helical coil having its adjacent windings in axial adjacency in accordance with microcoil-type vaso-occlusive devices of the prior art and being loaded in axial bending;

FIG. 8B is a schematic cross-sectional elevation view of a helical coil similar to that shown in FIG. 8A, except having its windings spaced at twice the pitch of the former, and being loaded in axial bending;

FIG. 8C is a schematic cross-sectional elevation view of a plurality of the double-pitched helical coils shown in FIG. 8B being loaded in parallel axial bending;

FIG. 8D is a schematic cross-sectional elevation view of the plurality of double-pitched helical coils shown in FIG. 8C combined such that their respective windings are in alternating, uniaxial adjacency, and being loaded in parallel, axial bending;

FIG. 9A is a schematic cross-sectional elevation view of a stack of O-rings being loaded in axial compression;

FIG. 9B is a schematic cross-sectional elevation view of a single helical coil having windings of a size similar to that of the O-rings of FIG. 9A and arranged in axial adjacency in accordance with microcoil-type vaso-occlusive devices of the prior art, and being loaded in axial compression;

FIG. 9C is a schematic cross-sectional elevation view of a plurality of helical coils similar to those of FIGS. 7D and 8D combined such that their respective windings are arranged in alternating, uniaxial adjacency, and being loaded in axial compression;

FIG. 10 is a partial cross-sectional elevation view of another exemplary preferred embodiment of a microcoil-type vaso-occlusive device in accordance with the present invention;

FIG. 11 is an enlarged cross-sectional axial view of the novel device of FIG. 10, as revealed by the section taken therein along the lines 11-11;

FIG. 12 is an enlarged cross-sectional elevation view of the novel device of FIG. 10 showing the relationship of the respective windings thereof, as revealed by the section taken in FIG. 11 along the lines 12-12;

FIG. 13A is a perspective view of eight filars being conjointly wound into helical coils on a mandrel, with their respective windings arranged in alternating, uniaxial abutment in accordance with a method for making one exemplary embodiment a vaso-occlusive device in accordance with the present invention;

FIG. 13B is a perspective view of four filars being conjointly wound into helical coils on a mandrel, with their respective windings arranged in a spaced-apart, alternating, axial adjacency in accordance with a method for making another exemplary embodiment of a vaso-occlusive device in accordance with the present invention;

FIG. 13C is a perspective view of four filars being conjointly wound into helical coils on a mandrel, with their respective windings arranged in alternating, uniaxial adjacency and in spaced-apart groups of four in accordance with a method for making another exemplary embodiment of a vaso-occlusive device in accordance with the present invention; and,

FIGS. 14A-14C are successive partial cross-sectional elevation views showing the axial buckling of an unrestrained vaso-occlusive device in accordance with the present invention upon encountering a wall generally transverse to its direction of axial movement.

### DETAILED DESCRIPTION OF THE INVENTION

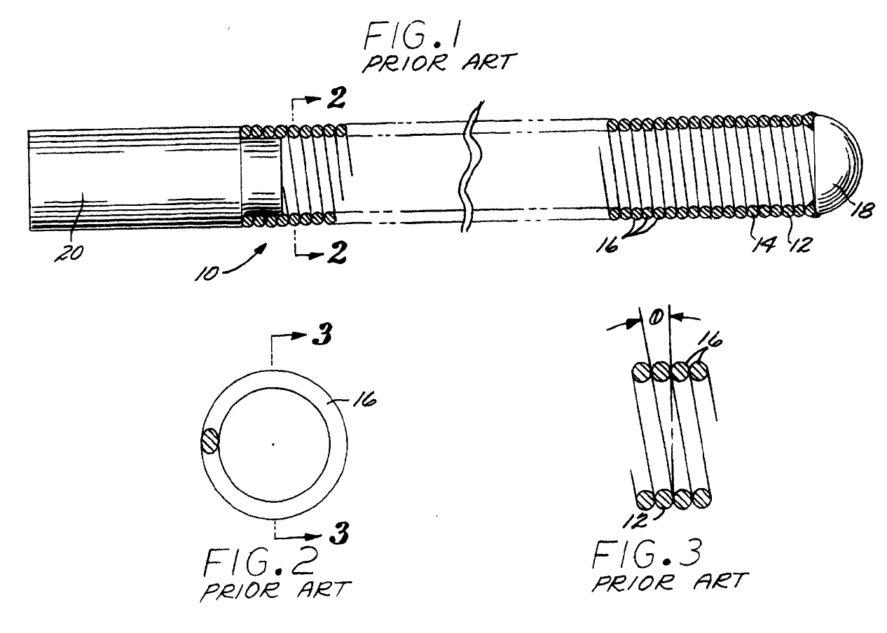

FIGS. 1-3 are partial cross-sectional elevation, enlarged cross-sectional axial, and enlarged cross-sectional elevation views, respectively, of a conventional microcoil-type vaso-occlusive implant device 10 in accordance with the prior art. As illustrated, the device 10 comprises a single helical coil 12, typically comprising a single resilient filar 14 helically wound such that the windings 16 of the resulting coil are in sequential, uniaxial adjacency or abutment with one another when the coil is in a relaxed state. That is, the windings 16 of the coil have an advance, or pitch, about equal to the cross-sectional axial dimension of the filar 14, typically a diameter “d” for filars having a round cross section. As may be seen in the enlarged view of FIG. 3, this arrangement results in a relatively small pitch angle, or “helical angle,” of φ, i.e., the angle that the windings 16 make with an axis perpendicular to the long axis of the device 10.

The conventional implant device 10 may also include a rounded ball tip 18 attached to the proximal end of the device, and a coupler 20 attached to a distal end thereof The distal end ball tip 18 functions as an obturator to facilitate atraumatic navigation of the implant 10 through tortuous vasculature during deployment of the device, and the proximal end coupler 20 provides a means for releasably attaching the device to a delivery mechanism (not shown), e.g., a delivery wire, and is detached with the device 10 once its placement in a target vascular site is effected. Additionally, the conventional implant device 10 may be provided with an axially recurvate, secondary “memory” shape (not illustrated) formed in the microcoil during its manufacture by, e.g., heat treatment, which the device will assume when relaxed and axially unconfined by the interior walls of, e.g., a catheter.

While the conventional implant device 10 illustrated has achieved some success in the field of vascular embolization, it has certain relevant mechanical properties that need improvement, including 1) its lack of resistance to axial stretching, 2) its resistance to axial bending, and 3) its resistance to axial buckling.

Regarding its lack of resistance to stretching, it may be seen that the conventional device 10 can be withdrawn axially from a given location by exerting a retrograde, or pulling, axial force on the proximal end of the device, ie., in a direction opposite to the distal end. However, if there is sufficient resistance to movement of the distal end of the device 10 from surrounding vasculature, the device is subject to an inelastic axial elongation and kinking. Such permanent deformation of the device 10 can render it unusable, necessitating its complete withdrawal from the patient and replacement with a new device.

Regarding the resistance of the device 10 to axial bending, it should be understood that the typical configuration of the device when fully deployed in the target cavity is a recurvate ball, or bundle, of windings. Thus, a lower resistance to axial bending aids not only in the deployment of the device 10 through tortuous vasculature, but also in the recurvation of the device within the target cavity.

Regarding the resistance of the device 10 to axial buckling, it should be understood that, during insertion of the device into a target cavity, e.g., an aneurysm, and after its windings are no longer axially supported by a catheter, its distal end or tip will eventually contact the opposite wall of the target cavity in a generally non-parallel direction. When this contact occurs, it is highly desirable that the device immediately buckles axially and begins recurvation, such that the distal tip of the device is immediately deflected parallel to or away from the cavity wall, rather than penetrating into or through it, which can result in tissue damage.

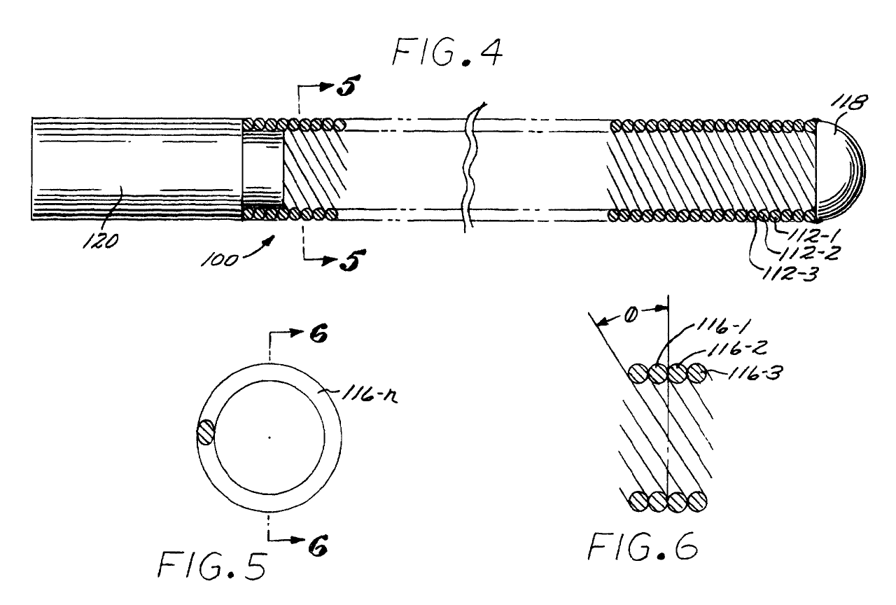

FIGS. 4-6 are partial cross-sectional elevation, enlarged cross-sectional axial, and enlarged cross-sectional elevation views, respectively, of a microcoil-type vaso-occlusive implant device 100 in accordance with a first exemplary embodiment of the present invention that advantageously addresses each of the above and other concerns of the prior art devices. As may be seen from a comparison of the respective sets of FIGS. 1-3 and 4-6, the novel device 100 has some features that are similar to the conventional device 10, and differs from it principally in the number n of helical coils 112-n incorporated therein, each comprising a helically wound filar 114-n, and the inter-arrangement of their respective windings 116-n with respect to each other. More particularly the novel implant device 100 comprises a multiplicity of filars 114-n which have been conjointly wound into elongate helical coils 112-n having their respective windings 116-n arranged in alternating, uniaxial adjacency with respect to each other when each of the respective coils is in a relaxed state.

The particular embodiment of implant device 100 illustrated in FIGS. 4-6 comprises three helical coils 112-1, 112-2, 113-3, each comprising a single, helically wound filar 114-n having the same, constant, round cross-sectional diameter d, and each of the respective windings 116-n of the coils has the same mean diameter Dₘ. Accordingly, in this particular embodiment, the pitch “P ” of the windings 116-n of the respective coils 112-n will be the same, and an integer multiple of, the number of coils n. It may be further noted by a comparison of FIGS. 3 and 6, that the pitch angle φ of the novel implant 100, which is given by tan⁻¹ (P/Dₘ) is substantially greater than that of the conventional implant 10, and in general, increases with the number of helical coils included in the device. Thus, as discussed below, the particular embodiment of implant device 100 illustrated in FIGS. 4-6 has about nine times the resistance to axial stretching as the prior art device 10 illustrated in FIGS. 1-3, about the same axial flexibility, or resistance to axial bending, and about a 300% reduction in the axial force necessary to initiate axial buckling, or distal tip deflection of the device, relative to that of the prior art device.

In a preferred embodiment, the multifilar device 100 is formed of between 2 and 9 filars 114-n of a biocompatible, radiopaque metal, such as platinum or a platinum-tungsten alloy, each having a cross-sectional diameter of about 0.05 mm, and accordingly, exhibits a pitch, or helical angle φ of between about 10° and 45°. This may be favorably contrasted with unifilar prior art devices 10, as shown in FIGS. 1-3, which typically have a pitch angle φ of between about 3° and 7°.

As further discussed below, it should be understood that the number of coils 112-n, the number, material, cross-sectional shape and sizes, or “gauges,” of their respective filars 114-n, and the pitch P and mean diameters Dₘ of their respective windings 116-n, can all be varied over a broad range to achieve desirable improvements in the mechanical properties of the implant 100, so long as the above “baseline” condition is observed, viz., that the respective windings 116-n of the coils 112-n be disposed in an alternating, “uniaxial” arrangement with respect to each other. This, in turn, implies that the respective windings 116-n of the coils 112-n turn in the same direction and have the same pitch angle φ, or stated alternatively, that none of the filars 114-n crosses over itself or another filar 114-n in the axial direction. Of course, it is possible to construct such “coaxial,” or “cross-ply,” devices, e.g., by making the outer diameters of some coils smaller than the inner diameters of others, and these devices are described in more detail in the above-referenced co-pending application Ser. No. ______, filed ______.

In another aspect, the material of the respective filars 114-n of the coils 112-n of the device 100 can be varied to provide other desirable properties therein. Thus, one or more filars 114-n can be made of a biocompatible metal or a polymer. In addition, the filars 114-n of each coil 112-n can each be made of a different material than that of the others. Indeed, each the coils 112-n may comprise one or more filars 114-n of any of a wide variety of materials, such as a radio-opaque material, including metals and polymers. Suitable metals and alloys include platinum, rhodium, palladium, rhenium, as well as tungsten, gold, silver, tantalum, and alloys of these metals. These metals have significant radiopacity, which aids in visualization of the device 100 during insertion and thereafter, and are also substantially biologically inert.

The filars 114-n of the coils 112-n may also be of any of a wide variety of stainless steels and other materials which maintain their shape despite being subjected to high stress, such as nickel/titanium alloys, preferably, the nickel/titanium alloy known as nitinol; platinum; tantalum; and various types of stainless steel that are known to be suitable for this type of application.

The filars 114-n may also be made of radiolucent fibers or polymers (or metallic threads coated with radiolucent or radiopaque fibers) such as Dacron (polyester), polyglycolic acid, polylactic acid, fluoropolymers (polytetrafluoroethylene), Nylon (polyamide), and silk. Should a polymer be used as the major component of the implant 100, it is desirably filled with some amount of a known radiopaque material, such as powdered tantalum, powdered tungsten, bismuth oxide, barium sulfate, and the like, for radio-visualization purposes.

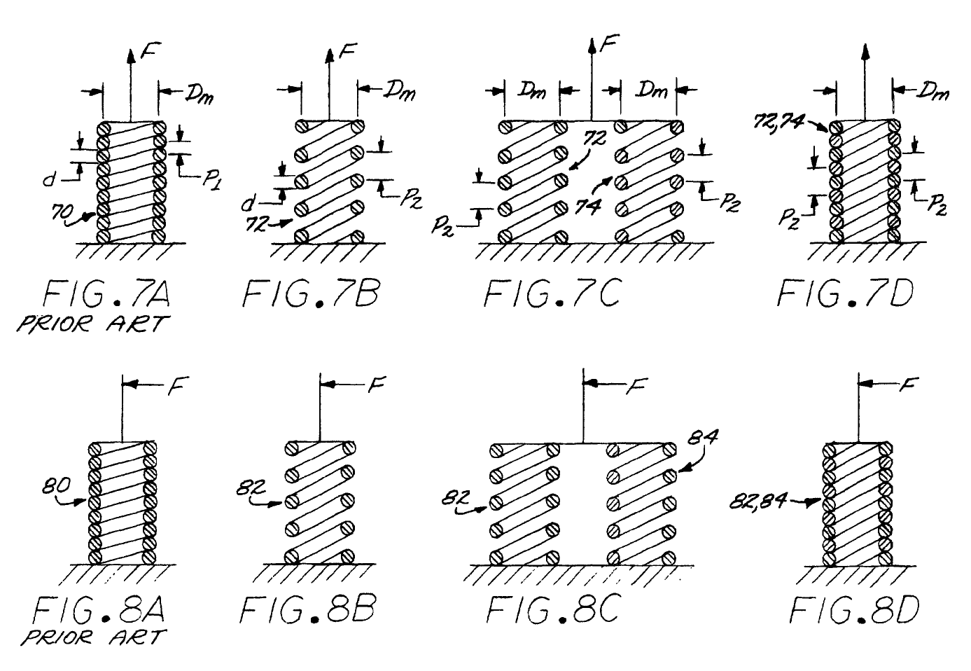

A better understanding of how the above improvements are obtained in the resistance to axial stretching, bending, and buckling of the implant 100 may be had from a consideration of the three sets of FIGS. 7A-7C, 8A-8C and 9A-9B, respectively.

FIG. 7A is a schematic cross-sectional elevation view of a single helical coil 70 having its windings in sequential, uniaxial adjacency in accordance with microcoil-type vaso-occlusive devices of the prior art, i.e., its comprises a single, helically wound filar having a round cross-section with a diameter d, and its windings have a mean diameter Dₘ and a pitch corresponding to the diameter d of the cross-section of its filar, ie., P₁=d. The coil 70 is being loaded in axial tension by a force F, i.e., the force F has a tendency to stretch the windings of the coil apart. If the force F exceeds the elastic yield point of the coil, the coil can become permanently elongated.

FIG. 7B is a view of another helical coil 72 identical to the coil 70 shown in FIG. 7A, except that its windings have twice the pitch of the latter, i.e., P₂=2P₁. Since the respective stiffness, or spring constant k, of the two coils is inversely proportional to the number of windings, or directly proportional to the pitch of the windings, of the respective coils, then if all other factors remain the same, the second coil 72 will have twice the axial stiffness, or resistance to axial stretching, as the first coil 70.

Thus, simply increasing the pitch, or spacing of the windings, of a microcoil will increase its resistance to axial stretching. However, this solution may be undesirable in some circumstances, e.g., when an acute radio-visualization of the coil is necessary. Thus, since the radiopacity of the coil is a function of the number of its windings, a coil with only half the number of windings will be only half as visible as a closely wound coil.

However, this drawback can be overcome by the addition of a third coil 74 to the second coil 72, as schematically illustrated in FIG. 7C. If the third coil 74 has the same coil pitch P₂ as the second coil 72, it will have the same stiffness k as the second coil, ie., twice that of the first coil 70, and the same radiopacity, i.e., half that of the first. Further, if the second and third coils 72 and 74 are loaded in parallel, their respective stiffnesses are additive, i.e., they are four times as stiff as the first coil 70 in combination, and hence, have four times the resistance to axial stretching in response to the same axial force F, and the same radiopacity. In general, similar coils, when so combined, will have a resistance to axial stretching that increases as the square of the number of coils n².

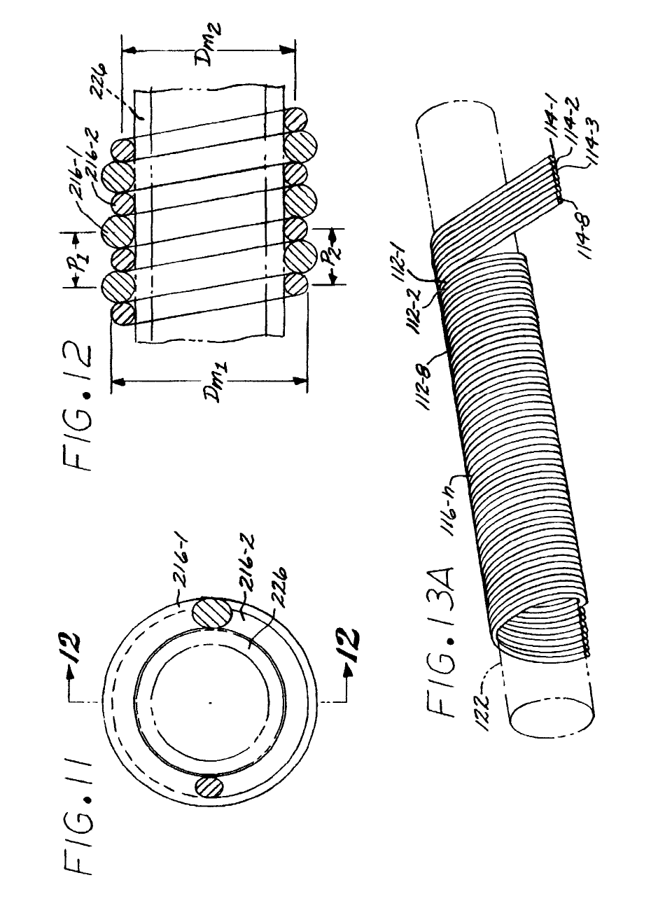

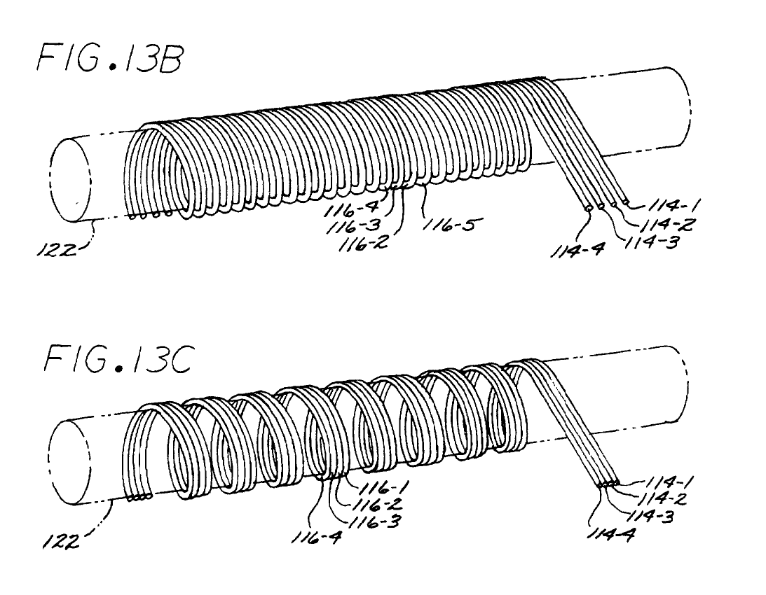

Finally, if the two double-pitched coils 72 and 74 are combined such that their respective windings are in alternating, uniaxial adjacency, such as is illustrated in FIG. 7D, the resulting combination will have the same number of windings, or radiopacity, as the first coil 70. This latter arrangement can be effected by screwing one of the two coils 72 and 74 into the other axially, or more preferably, by winding the two coils conjointly with each other, as illustrated in FIGS. 13A-13C.

FIG. 13A illustrates a multiplicity of filars 114-n being conjointly wound on a common support mandrel 122 (shown by phantom outline) into plurality of helical coils 112-n having their respective windings 116-n arranged in alternating, uniaxial abutment with each other. In the particular embodiment illustrated, eight filars 114-1-114-8 are shown being wound conjointly, but any number n of filars can be so wound. Alternatively, the filars 114-n can be conjointly wound using a “deflection winder” (not illustrated) of a known type, which forces the filars 114-n in parallel through a helical forming die simultaneously and thereby eliminates the need for a support mandrel 122.

FIG. 13B illustrates a similar winding method in which four filars 114-1-114-4 are being conjointly wound into four helical coils 112-n having their respective windings 116-n arranged in a uniaxial, alternating, axially spaced arrangement.

FIG. 13C illustrates a similar winding method in which a group of four filars 114-1-114-4 is being conjointly wound into four helical coils 112-n having their respective windings 116-n arranged in uniaxial, alternating, axially spaced groups of four windings each.

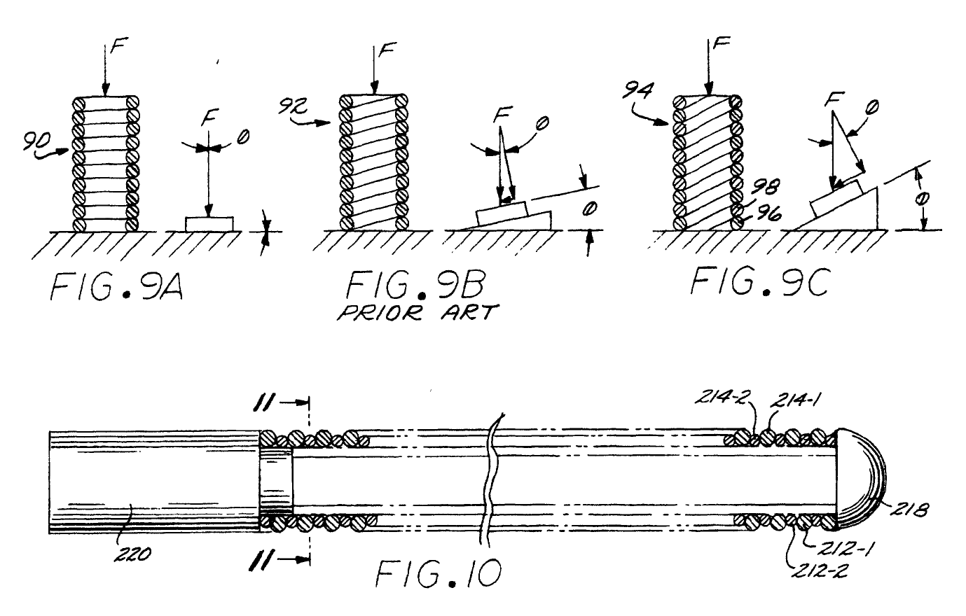

Turning now to the bending stiffness of the implant 100, three sets of helical coils 80, 82, and 84 are shown in FIGS. 8A-8C that are respectively identical to the coils 70, 72 and 74 illustrated in FIGS. 7A-7C, except that they are being loaded with a bending force F applied to one end thereof. Thus, the first coil 80 has the same filar diameter d and coil diameter Dₘ, and pitch P₁ as the first coil 70 of FIG. 7A, whereas, the second and third coils 80 and 82 are respectively identical to the first coil 80, except they each have twice the pitch P₂ thereof, i.e., P₂=2P₁.

Since the axial bending stiffness, or resistance, of a helical coil is directly proportional to the number of windings per unit length, or inversely proportional to the pitch P thereof, the second coil 82 will desirably have half the resistance to axial bending as the first coil 80. Thus, simply increasing the pitch, or spacing, of the windings of a microcoil will decrease its resistance to axial bending. However, as above, in some circumstances, this solution may be undesirable. But a third coil 84 can be combined in parallel with the second coil 82, as in the stretching discussion above and illustrated in FIG. 8D, and the combination of the two coils will have twice the radiopacity and bending resistance of the second coil 82, i.e., the same bending resistance and radiopacity as the first coil 80. Thus, any number of coils can be so combined in a device 100 without increasing its resistance to axial bending. On the other hand, adding coils does not desirably decrease the resistance of the device 100 to axial bending, and accordingly, if it is desirable to decrease the resistance of the device to axial bending, a tradeoff must be made between increasing the resistance of the device to axial stretching, and decreasing the resistance of the device to axial bending.

One way of achieving such a tradeoff is by varying other parameters of the respective coils of the implant device, such as the cross-sectional dimension dᵢ of their respective filars and/or the mean diameter Dₘ₁ of their respective windings. A second exemplary preferred embodiment of a microcoil-type vaso-occlusive implant device 200 in accordance with the present invention that incorporates such variations is illustrated in the partial cross-sectional elevation, enlarged cross-sectional axial, and enlarged cross-sectional elevation views of FIGS. 10-12, respectively. The second exemplary preferred embodiment of the device 200 incorporates two helical coils 212-1 and 212-2 with respective filars 214-1 and 214-2 having round cross-sections of differing diameters, ie., d₁>d₂, differing coil pitches, i.e., P₁>P₂, and differing mean coil diameters, viz., Dₘ₁>Dₘ₂. In the particular embodiment of device 200 illustrated, the respective windings 216-1 and 216-2 of the two coils 212-1 and 212-2 are wound such that their respective inner diameters are the same, for example, around a mandrel of a constant diameter.

The effects of making the respective filar and mean coil diameters d₂ and Dₘ₂ of the second coil 212-2 smaller than those of the first coil 212-1 are, on the one hand, to reduce the effective coil pitch of the combined device 200, and hence, its resistance to axial stretching, to a value somewhat less than 4 times that of a prior art, unifilar device, and on the other hand, to reduce the effective cross-sectional area of the device 200 in bending, and hence, its resistance to axial bending, to a value somewhat less than that of the prior art device. Thus, the device 200 trades off some increase in its resistance to axial stretching for a corresponding reduction in its resistance to axial bending, both respective values being substantially improved over those of the prior art device 10.

Another benefit of the second embodiment of the implant device 200 illustrated in FIGS. 10-12 that is not available in the prior art device 10 relates to the frictional resistance encountered by the device in sliding through a tubular catheter (not illustrated). Thus, it may be seen that the alternating size of the outer diameters of the windings 216-1, 216-2 of the device 200 imbue it with a substantially reduced effective outer surface area in contact with the inner surface of the catheter, as compared to the prior art device 10, wherein the windings 16 have the same outer diameter. Thus, the implant device 200 will slide through a catheter with much less frictional resistance than the prior art device 10.

Yet another advantage of the implant devices of the present invention over prior art implant devices is also illustrated in FIGS. 10-12, and relates to the hollow axial lumen 224 defined in the device 200 by the plurality of uniaxial helical coils 216-1 and 216-2. A flexible hollow tube or porous sponge 226 (shown by dashed outlines) can optionally be inserted into the lumen and used as a reservoir for the delivery of therapeutic agents, e.g., medications, for delivery thereof to a patient via the device. While such a tube or sponge 226 may increase the bending resistance of the device 200 slightly, it may be seen that this additional option is not available in those prior art devices that achieve increased stretching resistance by tying the two opposite ends of the device together with an axial strand through the center of the device, as this latter structure effectively blocks the lumen 224 and prevent the insertion of a tube or sponge therein.

The effect on a microcoil's resistance to axial buckling by additional coils is illustrated schematically in FIGS. 9A-9C. As will be appreciated by those of skill in the art, a microcoil-type vaso-occlusive device approximates a long, slender “column” in axial compression, and consequently, the classical mathematical relationships for analyzing its buckling characteristics can be quite complex. However, from a heuristic standpoint, a stack 90 of O-rings can be thought of as approximating a long, slender helical coil having a “pitch,” or helical angle φ, of zero, as illustrated in the small inset figure of FIG. 9A. As is known, such a stack 90 is “neutrally” stable when acted on by a purely vertical compressive force F as illustrated in FIG. 9A, and provided that the force F does not include any horizontal component, is capable of sustaining a substantially large value of the compressive force F without axial buckling.

FIG. 9B schematically illustrates the effect of inclining a stack 92 of O-rings by an angle of φ relative to the horizontal, as illustrated be the inset figure therein, and thus, approximating a single helical coil having its windings in adjacent uniaxial abutment, i.e., a prior art microcoil having pitch of I filar diameter d. The effect of increasing the pitch angle φ to a non-zero value is to convert a portion of the vertically acting force F into a component (F sin φ) acting parallel to the plane of the O-rings. As will be appreciated, such a stack 92 is conditionally unstable, and will collapse, or buckle axially, when the magnitude of this parallel force component is sufficient to overcome the frictional sliding forces acting between the respective O-rings.

FIG. 9C illustrates the effect on the axial buckling of a stack 94 of O-rings when the pitch angle φ is increased even further, e.g., by combining a pair of double-pitched helical coils 96 and 98 such that their respective windings are in alternating uniaxial abutment, in accordance with the present invention. As may be seen in FIG. 9C, the effect of such a combination is to double the pitch angle φ relative to the “single-coil” stack 92 shown in FIG. 9B, thereby increasing the component of the force F acting to overcome the frictional sliding resistance between the O-rings and rendering the stack more prone to axial buckling. In general, the force necessary to initiate axial buckling of the stack of O-rings varies as the cotangent of φ, and thus, decreases geometrically with increasing pitch angles φ. Of course, it should be understood that the adjacent windings of helical coils are actually connected to each other through more than just the frictional forces exerted between them alone, and therefore need a correspondingly greater force to initiate axial buckling, but the principals involved are the same.

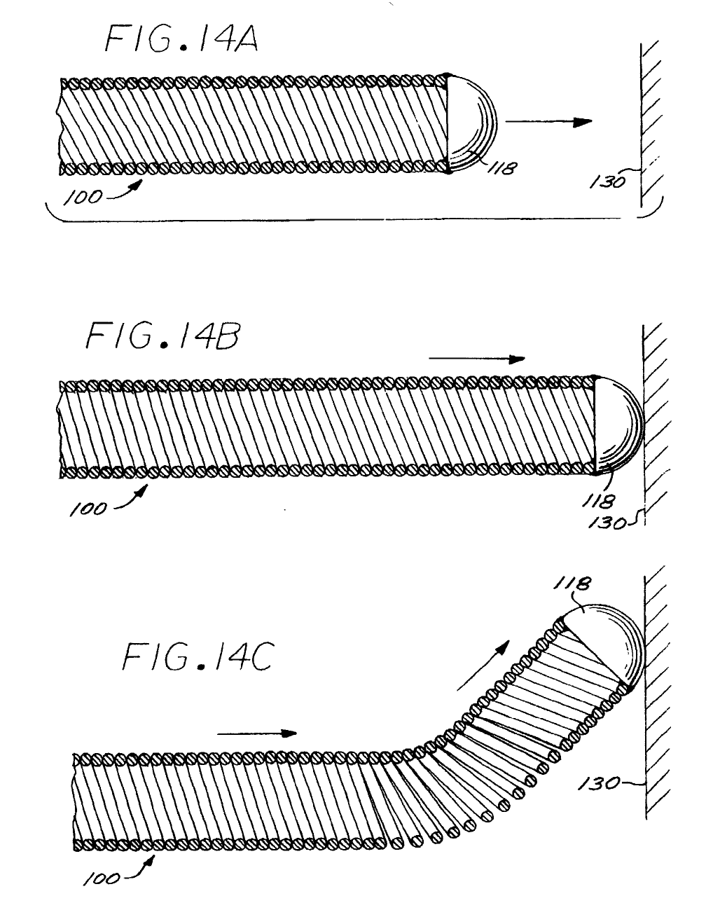

FIGS. 14A-14C sequentially illustrate the improved axial buckling of the first exemplary embodiment of multi-helix vaso-occlusive device 100 described above and illustrated in FIGS. 4-6. In FIG. 14A the device 100 is shown after leaving the axial support of an insertion catheter (not illustrated) and approaching a transverse wall 130, e.g., the wall of an aneurysm, in a non-parallel direction. FIG. 14B illustrates the axial configuration of the distal tip portion of the device 100 just as the distal ball tip 118 of the device contacts the transverse wall 130. FIG. 14C illustrates the configuration of the distal tip portion of the device 100 just after commencement of axial buckling thereof. As described above, the device 100 comprises three helical coils 112-1-112-3, and accordingly, requires approximately 300% less relative axial force applied by the wall 130 to the device to effect deflection of the distal tip than that required by the prior art device 10 for buckling.

The present invention thus exhibits several advantages over typical unifilar microcoil-type vaso-occlusive devices. For example, the implants 100 and 200 provide increased stretch resistance and axial buckling without sacrificing flexibility, and do so with materials that are already known and approved for use in vascular implants. Furthermore, implants constructed in accordance with the invention allow the implant to elongate slightly, to provide an indication that abnormal friction has been encountered, or that the device is knotted or trapped, while excessive elongation that would permanently stretch or kink the device is resisted.

While specific embodiments of the invention have been described herein, it will be appreciated that many variations and modifications will suggest themselves to those of ordinary skill in the art. For example, although the invention is described herein in the context of a vascular implant, it may be easily modified for use in occluding other bodily lumens, orifices, and passages. As a specific example, without limitation, the invention may be readily adapted for occluding a fallopian tube for sterilization purposes.

In another variation, the lumen of the devices can incorporate an elongate body of an expansile, hydrophilic polymer, i.e., “hydrogel,” to further enhance the occlusive properties of the device.

Accordingly, the scope of the present invention should not be limited by the specific embodiments thereof described and illustrated herein, as these are merely exemplary in nature. Rather, the scope of the invention should be commensurate with that of the claims appended hereafter, and their functional equivalents.

What is claimed is:

1. A vaso-occlusive device, comprising a multiplicity of elongate filars conjointly wound into respective helical coils having respective windings arranged in alternating, uniaxial adjacency.

2. The device of claim 1, wherein the coils are connected in parallel.

3. The device of claim 1, wherein the respective windings of each coil have about the same mean diameter and axial pitch.

4. The device of claim 3, wherein the number of filars comprises between about 2 and 9, and wherein the axial pitch comprises between about 10° and 45°.

5. The device of claim 1, wherein each filar has a uniform cross-sectional area.

6. The device of claim 5, wherein the cross-sectional area of each filar of each coil is about the same as that of the filars of the other coils.

7. The device of claim 1, wherein the filar of each coil has a round cross-section.

8. The device of claim 1, wherein the filar of at least one coil comprises a metal.

9. The device of claim 1, wherein the filar of at least one coil comprises a polymer.

10. The device of claim 1, further comprising a rounded ball tip at a distal end thereof

11. The device of claim 1, farther comprising a coupler at a proximal end thereof.

12. The device of claim 1, wherein the respective coils define an axial lumen, and further comprising a flexible tube or porous sponge disposed within the lumen.

13. In a vaso-occlusive device of a type that includes a single helical coil, the improvement comprising a second helical coil wound conjointly with the first coil such that respective windings of the two coils are in alternating, uniaxial adjacency.

14. The device of claim 13, wherein the two coils are connected in parallel.

15. The device of claim 13, wherein the respective windings of the two coils have about the same mean diameters.

16. A method for making a vaso-occlusive device, the method comprising:
providing a multiplicity of elongate filars; and,
conjointly winding the filars into helical coils in which the respective filars are disposed in alternating, uniaxial adjacency to each other.

17. The method of claim 16, further comprising respectively connecting at least one set of respective proximal and distal ends of the filars together.

18. The method of claim 16, wherein conjointly winding the filars comprises winding the filars around a mandrel.

19. The method of claim 16, wherein conjointly winding the filars comprises winding the filars with a deflection winder.

20. The method of claim 16, further comprising attaching at least one of an obturator and a coupling at a respective one of a distal end and a proximal end of the filars.
